(19) United States
(12) Patent Application Publication
(10) Pub. No.: US 2013/0230738 A1
(43) Pub. Date: Sep. 5, 2013

(54) POROUS IMPLANT MATERIAL

(71) Applicants: Yuzo Daigo, Kitamoto-city (JP); Shinichi Ohmori, Kitamoto-city (JP); Komei Kato, Saitama-shi (JP)

(72) Inventors: Yuzo Daigo, Kitamoto-city (JP); Shinichi Ohmori, Kitamoto-city (JP); Komei Kato, Saitama-shi (JP)

(73) Assignee: MITSUBISHI MATERIALS CORPORATION, Tokyo (JP)

(21) Appl. No.: 13/884,129

(22) PCT Filed: Nov. 10, 2011
(86) PCT No.: PCT/JP2011/075949

§ 371 Date: May 8, 2013

(30) Foreign Application Priority Data
Nov. 10, 2010 (JP) 2010-251431

(51) Int. Cl. A61L 27/04; A61C 8/00; A61L 27/56; A61F 2/44

(52) U.S. Cl. CPC A61L 27/04; A61F 2/442; A61C 8/0012; A61L 27/56
USPC 428/613

(57) ABSTRACT

Providing porous implant material having a strength property approximate to human bone, without arising stress shielding, and which is possible to maintain sufficient bound strength with human bone. Porous implant material according to the present invention has a plurality of porous metal bodies 4 which are bonded with each other at bonded-boundary surface F parallel to a first direction, wherein each the porous metal body has a three-dimensional network structure formed from a continuous skeleton 2 in which a plurality of pores 3 are interconnected, a porosity rate is 50% to 92%, and a compressive strength compressing in a direction parallel to the bonded-boundary surface F is 1.4 times to 5 times of a compressive strength compressing in a direction orthogonal to the bonded-boundary surface F.

Priority is claimed on Japanese Patent Application No. 2010-251431, filed Nov. 10, 2010, the content of which is incorporated herein by reference.

### BACKGROUND OF THE INVENTION

1. Field of the Invention

The present invention relates to material used for an implant implanted intravitally, and in particular, relates to implant material made of porous metal.

2. Description of the Related Art

Patent Documents 1 to 3 describes implants which are implanted intravitally.

An implant (an intervertebral spacer) described in Patent Document 1 is used by inserted and arranged between centrums from which an intervertebral disk is removed. In order to easily insert the implant and prevent the implant from falling out, the implant is includes a spacer body with an upper surface and a lower surface having unique figures.

An implant (a dental implant) described in Patent Document 2 is formed from: a heart material which is formed from solid-columnar titanium or titanium alloy; and a porous layer which is arranged by the heart material. The porous layer is made by sintering a plurality of spherical grains made of titanium or titanium alloy so that a plurality of continuous holes are made between the spherical grains which are bound with each other by sintering. The spherical grains each have a surface layer of gold-titanium alloy, so that the adjacent spherical grains are bound with each other by the surface layers. Accordingly, the implant described in Patent Document 2 is suggested as a small dental implant having high bound strength with a jawbone.

An implant described in Patent Document 3 is made of porous material, and includes a first part with high porosity rate and a second part with low porosity rate. In this case, for example, by inserting the second part of the implant made from absolute high-density material having a titanium-inlay-shape into a hole made at the second part of the implant having a shape of titanium foam in green and sintering them, the second part is adhered by contracting the first part. The second part with low porosity rate is used for implanting or adhesion, so that it can be prevented to waste the grains in implanting or adhesion because of the low porosity rate.

### PRIOR ART DOCUMENTS

### Patent Documents

Patent Document 1: Japanese Examined Patent Application, Second Publication No. 4164315
Patent Document 2: Japanese Examined Patent Application, Second Publication No. 4061581
Patent Document 3: Japanese Translation of the PCT International Publication, Publication No. 2009-504207

### SUMMARY OF THE INVENTION

### Problems to be Solved by the Invention

Since these implants are used for a part of intravital bone, excellent conherence to bone and appropriate strength for assuming a part of bone. However, the strength tends to fail if following the cohesion to bone; on the other hand, the cohesion to bone tends to be poor if following the strength, so that it is difficult to satisfy both of the strength and the cohesion.

The implants described in Patent Documents 2 and 3 are considered to be possible to satisfy the cohesion to bone and the necessary strength since they have a composite construction of the solid-heart material and the porous layer or a composite construction of the first part with high-porosity rate and the second part with low-porosity rate. However, if metal material is used as an implant, since metal material generally has higher strength than that of human bone, the implant may receive most of load on bone, so that stress shielding (i.e., a phenomena in which the vicinity of inserted part of the implant to bone becomes brittle) may arise.

Therefore, it is required for the implants to have the strength equivalent to that of the human bone. However, the human bone has a combined structure of bio-apatite having a dimetric crystal construction with collagen fiber, and has a strength property preferentially oriented along a C-axis direction. Accordingly, it is difficult for the implant to approach the human bone simply by combining the structures as described in Patent Documents.

The present invention is achieved in consideration of the above circumstances, and has an object to provide porous implant material having a strength property approximate to human bone, without arising stress shielding, and which is possible to maintain sufficient bound strength with human bone.

### Means for Solving the Problem

Porous implant material according to the present invention has a plurality of porous metal bodies which are bonded with each other at bonded-boundary surface parallel to a first direction, wherein each the porous metal body has a three-dimensional network structure formed from a continuous skeleton in which a plurality of pores are interconnected, a porosity rate is 50% to 92%, and a compressive strength compressing in a direction parallel to the bonded-boundary surface is 1.4 times to 5 times of a compressive strength compressing in a direction orthogonal to the bonded-boundary surface.

The porous implant material can be unitarily bonded to bone by infiltrating the bone into the interconnected pores. Furthemore, since the plurality of porous metal bodies are bonded, the compressive strength at the bonded-boundary surface in a direction along the bonded-boundary surface is higher than the compressive strength at a center part. Therefore, in total, the compressive strength in the direction along the bonded-boundary surface is higher the compressive strength orthogonal to the bonded-boundary surface, so that a strength property is anisotropic as human bone. Accordingly, by implanting the porous implant material into a human body with according the anisotropic strength to a directional strength property of human bone, the stress shielding can be efficiently prevented from arising.

In this case, if the porosity rate is lower than 50%, the filtration of bone is slow, so that a bound function is insufficient. If the entire porosity rate is higher than 92%, the compressive strength is low, so that function as an implant of supporting bone is insufficient.

By bonding the plurality of porous metal bodies, various block-like materials can be easily made; and it is possible to layer the porous metal bodies having different porosity rates from each other. Therefore, it is flexible to design the porous implant material; for example, while the entire porosity rate is maintained, the porosity rate can be partially different.

Furthermore, in a case in which the porous implant material formed as described above is utilized as an implant, it is possible to add a porous metal body which is bonded at a bonded-boundary surface with a different direction from the direction parallel to the first direction if required.

In the porous implant material according to the present invention, it is preferable that the porous metal bodies be foam metal made by expanding and sintering after forming expandable slurry containing metal powder and expanding agent so as to have higher metallic density at a front surface than at a center part.

The foam metal can be made so as to have the three-dimensional network structure of the continuous skeleton and the pores, and can be controlled in the porosity rate at a wide range by foam of the expanding agent. Therefore, the foam metal can be appropriately utilized according as an intended part.

Moreover, in the foam metal, an opening rate at a surface can be controlled independently of the entire porosity rate. Therefore, by raising a metallic density at the surface (i.e., reducing the opening rate), strength of the bonded-boundary surface can be improved.

In the porous implant material of the present invention, it is preferable that the pores be formed to have flat shapes which are long along a direction parallel to the bonded-boundary surface and short along a direction orthogonal to the bonded-boundary surface, and a length along the bonded-boundary surface be 1.2 times to 5 times of a length orthogonal to the bonded-boundary surface.

Since the pores are formed in flat, the compressive strength in the flat direction is higher than the compressive strength in the orthogonal direction. Therefore, by forming the pores in flat so as to long along the bonded-boundary surface, the compressive strength in the direction along the bonded-boundary surface can be improved. Specifically, since the pores are formed so that the flat direction is along the bonded-boundary surface, the compressive strength in the direction along the bonded-boundary surface can be improved. Furthermore, since the length along the bonded-boundary surface is 1.2 times to 5 times of the length orthogonal to the bonded-boundary surface, the compressive strength in the direction parallel to the bonded-boundary surface can be efficiently improved.

In the porous implant material of the present invention, part of the plurality of porous metal bodies have different porosity rate from the other porous metal bodies.

By bonding the porous metal bodies having the different porosity rates, while the entire strength can be maintained, and it is easy for the bone to be infiltrated in the porous metal body with high porosity rate.

### Effects of the Invention

According to the porous implant material of the present invention, since bonding the porous metal bodies, the compressive strength in the direction along the bonded-boundary surface can be higher than the compressing strength in the direction orthogonal to the bonded-boundary surface. Moreover, since the porous implant material has the strength property with anisotropic near to human bone, by utilizing the porous implant material with according the anisotropic strength to the direction of bone, the stress shielding can be efficiently prevented from arising. Furthermore, it is easy for bone to infiltrate by the interconnected pores, so that the cohesion to bone can be sufficiently maintained.

### BRIEF DESCRIPTION OF THE DRAWING

FIG. 1 is a perspective view schematically showing an embodiment of porous implant material according to the present invention.

FIG. 2 is a schematic view showing a cross-section of porous metal bodies in the porous implant material shown in FIG. 1.

FIG. 3 is a schematic structural view showing a forming apparatus for producing the porous metal bodies.

FIG. 4 is a schematic view showing a cross-section of porous metal bodies in the porous implant material of another embodiment.

FIG. 5 is a photo image by an optical microscope showing a front surface of the porous implant material of an example.

FIG. 6 is a photo image by an optical microscope showing a cross section of the porous implant material of an example.

FIG. 7 is a graph showing a distribution of pore diameters in the porous implant material of examples.

FIG. 8 is a graph showing degrees of dependence of compressive strengths on porosity rates and pore-shapes.

FIG. 9 is a perspective view showing another embodiment of the present invention.

FIG. 10 is a plan view showing another embodiment of the present invention.

FIG. 11 is a plan view showing another embodiment of the present invention.

FIG. 12 is a perspective view showing another embodiment of the present invention.

FIG. 13 is a schematic structural view showing a substantial part of another forming apparatus for producing the porous metal bodies.

### DETAILED DESCRIPTION OF THE INVENTION

Below, embodiments of porous implant material according to the present invention will be explained with reference to drawings.

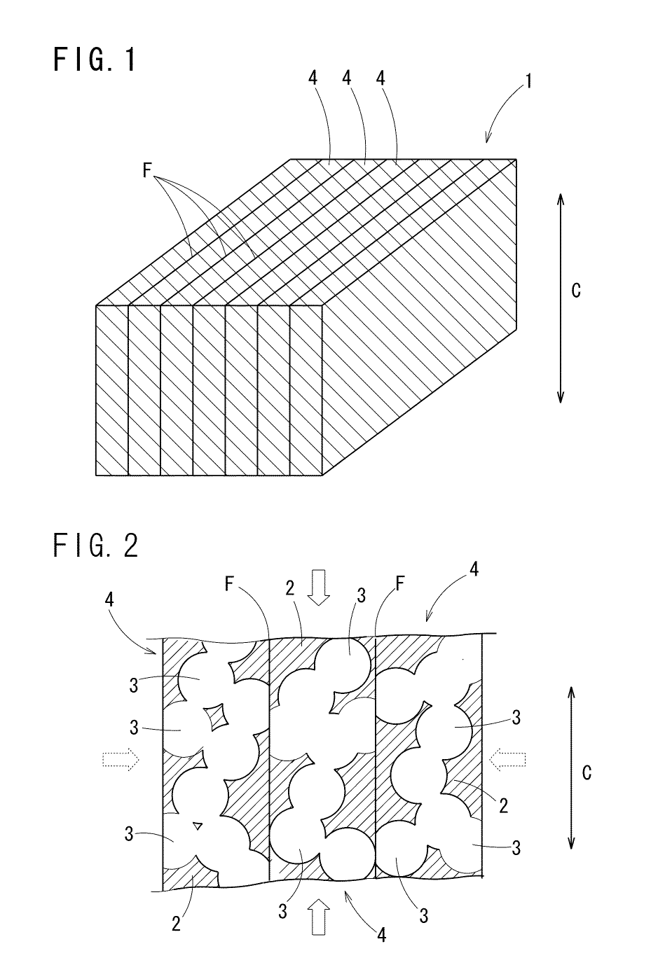

As shown in FIG. 1 and FIG. 2, porous implant material 1 of the present embodiment is made by laminating, a plurality of plate-like porous metal bodies 4 of foam metal having three-dimensional network structure formed from a continuous skeleton 2 in which a plurality of pores 3 are interconnected, at bonded-boundary surfaces F which are parallel to each other. The foam metal is made by expanding and sintering after forming expandable slurry containing metal powder and expanding agent and the like into a sheet-shape as described later. In the foam metal, the pores 3 are open at a front surface, a back surface, and a side surface. The foam metal is made close at the vicinity of the front surface and the back surface with respect to a center part of a thickness direction.

The porous implant material 1 made by laminating the porous metal bodies 4 of the foam metal has an entire porosity of 50% to 92%.

In the porous implant material 1, a first direction along the bonded-boundary surface F of the porous metal bodies 4 is set to an axial direction C when implanting into a living body. In FIG. 1 and FIG. 2, the axial direction C is set vertical.

Next, a producing method of the porous implant material 1 will be explained.

The porous metal bodies 4 forming the porous implant material 1 are produced by forming expandable slurry containing metal powder, expanding agent and the like into a sheet-shape by Doctor Blade method or the like, dehydrating the sheet so as to make a green sheet, and expanding the green sheet after a degreasing process and a sintering process. Furthermore, by layering and sintering a plurality of green sheets, the porous implant material 1 in which the porous metal bodies 4 are layered is produced.

The expandable slurry is obtained by kneading metal powder, binder, plasticizer, surfactant, and expanding agent with water as solvent.

As metal powder, for example, powder of metal or oxide thereof which is biologically innocuous for is used, such as pure titanium, titanium alloy, stainless steel, cobalt chromium alloy, tantalum, niobium, or the like. These powders can be produced by hydrogenate-dehydrogenate method, atomize method, chemical process method or the like. An average particle size of these powders is preferably 0.5 μm to 50 μm. These powders are contained in the slurry at 30% by mass to 80% by mass.

As the binder (i.e., a water-soluble resin binder), methyl cellulose, hydroxypropyl methylcellulose, hydroxyethyl methylcellulose, carboxymethylcellulose ammonium, ethyl cellulose, polyvinyl alcohol or the like can be used.

The plasticizer is added in order to plasticize a compact obtain by forming the slurry. As the plasticizer, for example, polyalcohols such as ethylene glycol, polyethylene glycol, glycerin and the like, oils and fats such as sardine oil, rapeseed oil, olive oil and the like, ethers such as petroleum ether and the like, and esters such as diethyl phthalate, di-n-butyl phthalate, diethylhexyl phthalate, dioctyl phthalate, sorbitan monooleate, sorbitan trioleate, sorbitan palmitate, sorbitan stearate and the like can be used.

As the surfactant, anion surfactants such as alkyl benzene sulfonate, α-olefin sulfonate, alkyl ester sulfate, alkyl ether sulfate, alkane sulfonate and the like, nonionic surface-active agent such as polyethylene glycol derivatives, polyhydric alcohol derivatives and the like, and ampholytic active agent and the like can be used.

As the expanding agent, agent which can form pores in the slurry by generating gas can be used. For example, volatile organic solvents such as pentane, neopentane, hexiane, isohexane, isoheptane, benzene, octane, toluene and the like, that is, anti-soluble hydrocarbon-system organic solvent having carbon number of 5 to 8 can be used. It is preferably that the expanding agent be contained in the expandable slurry by 0.1 to 5% by weight.

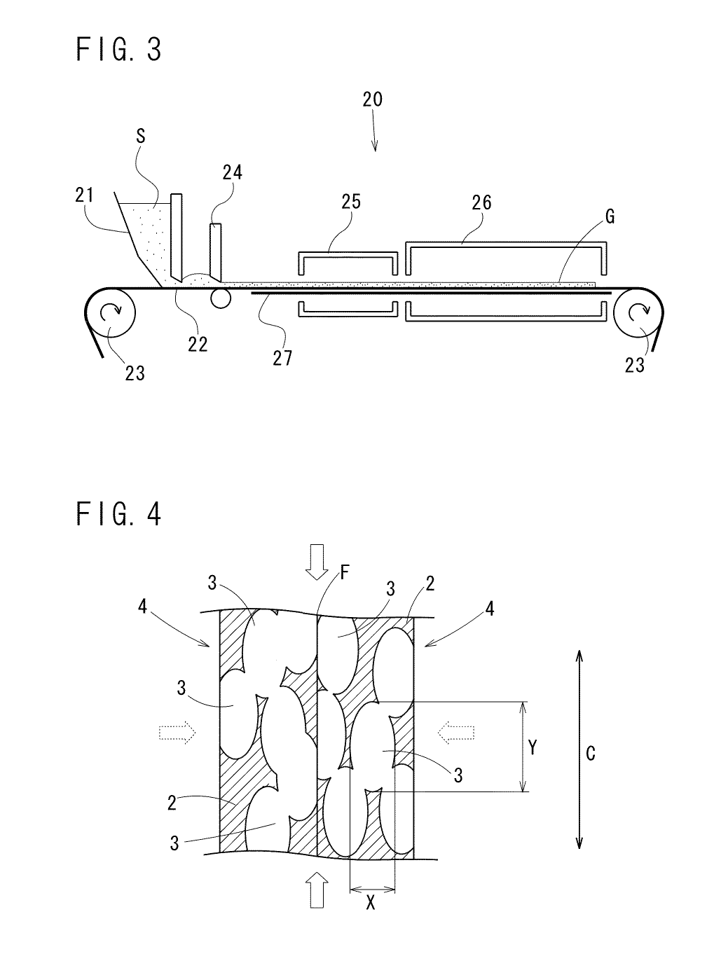

The green sheet is formed for the porous metal bodies 4 using the forming apparatus 20 shown in FIG. 3 from the expandable slurry S prepared as described above.

The forming apparatus 20 forms a sheet by Doctor Blade Method, is provided with: a hopper 21 in which the expandable slurry S is stored; a carrier sheet 22 transferring the expandable slurry S supplied from the hoper 21; rollers 23 supporting the carrier sheet 22; a blade (a doctor blade) 24 forming the expandable slurry S on the carrier sheet 22 at a prescribed thickness; a constant-temperature high-humidity chamber 25 in which the expandable slurry S is expanded; and a dehydrate chamber 26 in which the expanded slurry is dehydrated. A lower surface of the carrier sheet 22 is supported by a supporting plate 27.

Forming Process of Green Sheet

In the forming apparatus 20, at first, the expandable slurry S is charged in the hopper 21 so as to supply the expandable slurry S on the carrier sheet 22 from the hopper 21. The carrier sheet 22 is supported by the rollers 23 rotating to the right in the illustration and the supporting plate 27 so that an upper surface thereof is moved rightward in the illustration. The expandable slurry S supplied on the carrier sheet 22 is moved along with the carrier sheet 22, and formed into plate-shape by the blade 24.

Next, the plate-shape expandable slurry S is expanded in the constant-temperature high-humidity chamber 25 with a prescribed condition (ex., is 30° C. to 40° C. of temperature, 75% to 95% of humidity) with being moved for, for example, 10 minutes to 20 minutes. Subsequently, the expanded slurry S expanded in the constant-temperature high-humidity chamber 25 is dehydrated in the dehydrate chamber 26 with a prescribed condition (ex., 50° C. to 70° C. of temperature) with being moved for, for example, 10 minutes to 20 minutes. As a result, a spongiform green sheet G is obtained. A plurality of the green sheets G are produced.

Layering and Sintering Process

The green sheets G obtained as above are degreased and sintered in a state of being layered alternately so that the layered body of the porous metal bodies 4 is formed. Specifically, the binder (i.e., water-soluble resin binder) in the green sheets G are removed (degreased) under a condition in vacuum, 550° C. to 650° C. of temperature for 25 minutes to 35 minutes, and then further sintered under a condition in vacuum, 700° C. to 1300° C. for 60 minutes to 120 minutes.

The layered body of the porous metal bodies 4 as obtained above has three-dimensional network structures formed from continuous skeletons in which a plurality of pores 3 are interconnected. The porous metal bodies 4 are produced by foaming and sintering the green sheet G molded on the carrier sheet 22 so that densities at vicinities of a surface being in contact with the carrier sheet 22 and the counter surface thereof, that is, the densities at the vicinities of a front surface and a back surface, are closer than that of a center part along a thickness direction to have high metallic density. Therefore, the layered body has a closer density at the vicinity of the bonded-boundary surface (i.e., layered-boundary surface) F than that at, the center part between the bonded-boundary surface F. In the porous metal bodies 4, the pores 3 are open at the front surface and the back surface. Therefore, also in the layered body of the porous metal bodies 4, the pores 3 are interconnected from the front surface to the back surface.

A desired porous implant material 1 is obtained by cutting the layered body of the porous metal bodies 4 into an appropriate shape.

In the porous implant material 1 as produced above, owing to the porosity having the porosity rate of 50% to 92%, it is easy to infiltrate for bone when the porous material 1 is used as an implant, so that the cohesion to the bone is excellent. Moreover, as described above, since the density is hither at the vicinity of the bonded-boundary surfaces F than that at the center part between the bonded-boundary surfaces F, the strength when being compressed in the axial direction C parallel to the bonded-boundary surface F is higher than the strength when being compressed orthogonal to the bonded-boundary surface F (i.e., orthogonal to the axial direction C, that is the thickness direction of the porous metal bodies). Since the porous implant material 1 has thus the compressive strength with anisotropic and the strength property near to the human bone, the stress shielding can be efficiently prevented from arising. Specifically, it is preferable that the axial direction C along the bonded-boundary surfaces F of the porous implant material 1 agree with a C-axis direction of the bone.

The human bone is structured from a sponge bone at the center part thereof and a cortical bone surrounding the sponge bone. When the porous implant material is used as the sponge bone, the compressive strength in the axial direction C is preferably 4 to 70 MPa; and an elastic module of the compression is preferably 1 to 5 GPa. When the porous implant is used as the cortical bone, the compressive strength in the axial direction C is preferably 100 to 200 MPa, and the elastic module of the compression is preferably 5 to 20 GPa. In each case, it is preferable that the compressive strength in the axial direction C be directional so as to be 1.4 times to 5 times of the compressive strength of the compressive strength in the direction orthogonal to the axial direction C

In the above embodiment, the layered body of the porous metal bodies 4 is obtained by sintering the green sheets in a state of being layered. Furthermore, the layered body can be compressed in the thickness direction with a prescribed pressure by pressing or rolling.

FIG. 4 schematically shows a cross section in a state in which the layered body is pressed. The layered body of the porous metal bodies 4 is pressed in the thickness direction, the pores 3 are pressed so that the pores 3 have flat shape in which a length along the front surface (i.e., along the bonded-boundary surface F) is long and a length orthogonal to the front surface (i.e., in the thickness direction) is short. Accordingly, the compressive strength in the direction along the flat direction is higher than the compressive strength orthogonal to the flat direction, so that the directivity can be obtained by the strength property. In this case, if the length Y of the pores 3 along the bonded-boundary surface F is 1.2 times to 5 times of the length X orthogonal to the bonded-boundary surface F, the compressive strength in the direction parallel to the bonded-boundary surface F can be efficiently improved.

Moreover, it is preferable that the porous implant material 1 be implanted in the human body so that the first direction along the flat direction of the pores 3 (i.e., the first direction along the bonded-boundary surface F) agrees with the axial direction C.

### EXAMPLES

Below, examples of the porous implant material formed from the layered body of the porous metal bodies and the examples of the porous implant material in, a state in which the pores are pressed by compressing the layered body.

The green sheets were made by the expanding slurry method, and then the porous metal bodies were made from the green sheets. As material, metal powder of titanium having an average particle size of 20 μm, polyvinyl alcohol as a binder, glycerin as a binder, alkyl benzene sulfonate as surfactant, and heptane as expanding agent are kneaded with water as solvent, so that slurry was made. The slurry was formed into a plate-shape and dehydrated, so that the green sheets were made. Subsequently, the green sheets were layered, degreased and sintered, so that layered body of the porous metal bodies was obtained.

The implant material which was the layered body of the porous metal bodies as is and the implant material in which the layered body was rolled by the rolling machine were produced. As a comparative example, an implant material obtained by sintering one green sheet was also produced.

Then, with respect to the implant material obtained by pressing the layered body of the porous metal bodies, a front surface and a cross section in the thickness direction was observed by an optical microscope.

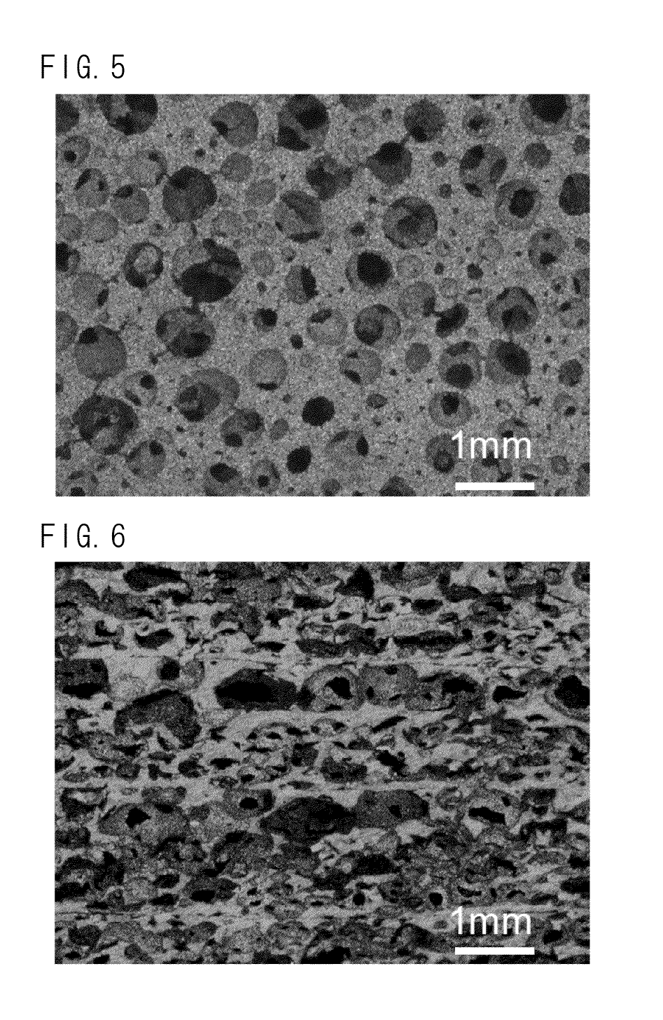

FIG. 5 is a photo image of the front surface. FIG. 6 is a photo image of the cross section. As is clear from those photo images, the pores opening at the front surface are substantially circular; at the cross section, the pores are pressed so as to be oblong in the thickness direction. Furthermore, it is recognized that the metal portion is close in the vicinity of the bonded-boundary surfaces.

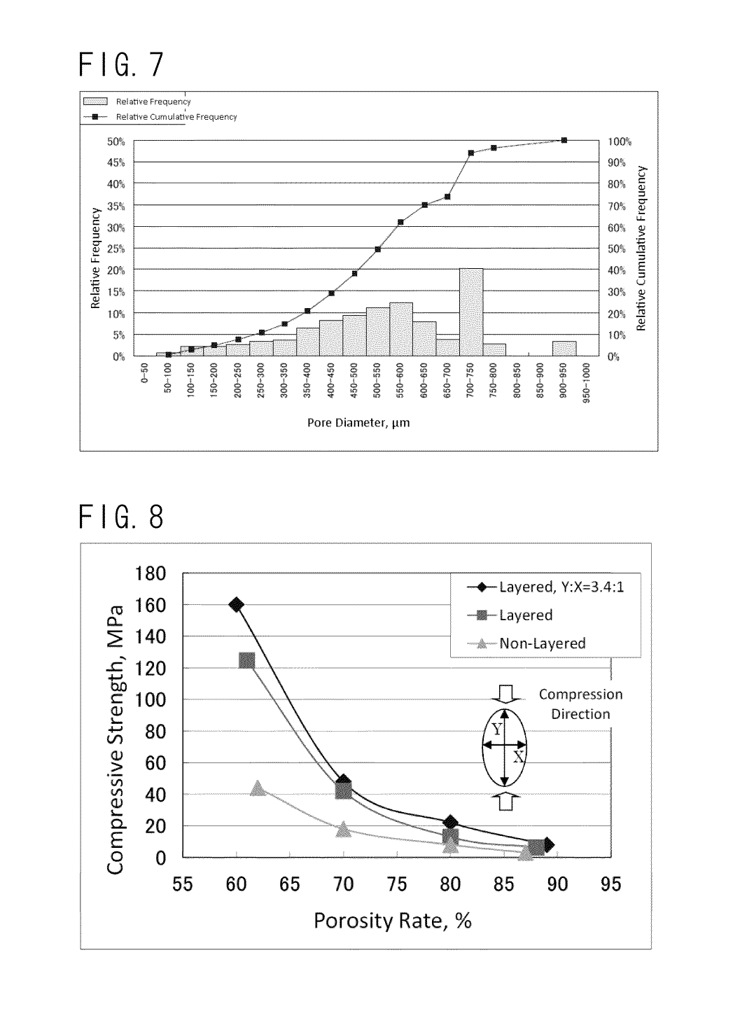

FIG. 7 is a graph showing a distribution of pore diameters. An average pore size at the front surface was substantially 550 μm, and an opening rate was substantially 60%.

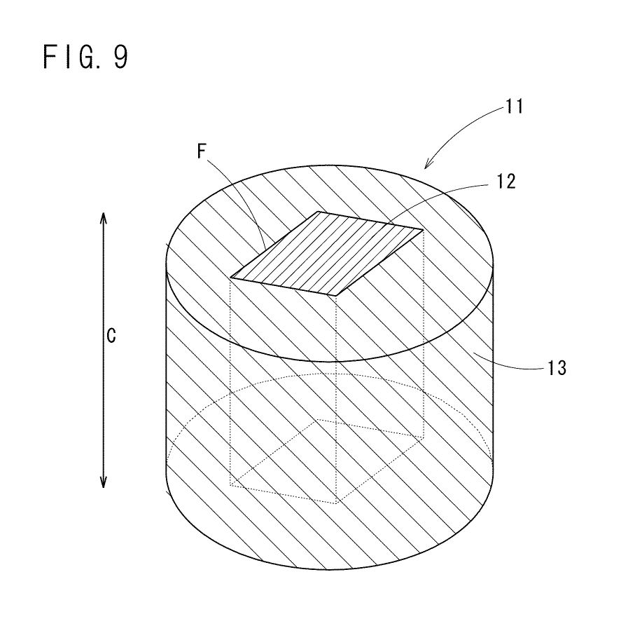

FIG. 8 is a graph showing degrees of dependence of the compressive strengths on the porosity rates and pore-shapes. With respect to the implant material obtained by pressing the layered body of the porous metal bodies, a prolate degree Y/X, that is, a ratio of the length Y along the bonded-boundary surface and the length X orthogonal to the bonded-boundary surface of the pores was measured. In this case, the prolate degree of the pores in each sample was obtained by: selecting five to ten pores in which the shapes thereof were easy to be certified in a photo image by the optical microscope; calculating the prolate degrees from the lengths Y and X of the selected pores from the photo image; and averaging the prolate degrees.

Then, the strengths were measured with adding compression load parallel to the front surface. In the pores which were flat by the compression, the compression load was parallel to a longitudinal direction of the pore. The compressive strengths were measured according to JIS H 7920 (Method for Compressive Test of Porous Metals).

In FIG. 8, the implant material which was the layered body of the porous' orous metal bodies as is denoted as “Layered”; and the implant material which was compressed was denoted as “Layered, Y:X=3.4:1”.

As shown in FIG. 8, in a case in which the implant material was the layered body and had the porosity rate of 70%, the compressive strength when compressed in the direction parallel to the bonded-boundary surface was 45 MPa. In a case in which the prolate degree was 3.4 (i.e., Y:X=3.4:1) and the porosity rate was 70%, the compressive strength when compressed in the direction parallel to the front surface was 48 MPa. Therefore, the strength when pressed in the direction along the front surface is about 1.7 times of the strength when pressed in the direction orthogonal to the front surface.

From the result shown in FIG. 8, it can be recognized that appropriate implants having a wide range of the compressive strength from the implant material obtained from the layered body of the porous metal bodies and the implant material obtained by compressing the layered body by adjusting the porosity rate appropriately.

The present invention is not limited to the above-described embodiments and various modifications may be made without departing from the scope of the present invention.

For example, if the plurality of porous metal bodies are layered, the porous metal bodies may have the same porosity rate; alternatively, the porous metal bodies having the different porosity rates can be layered.

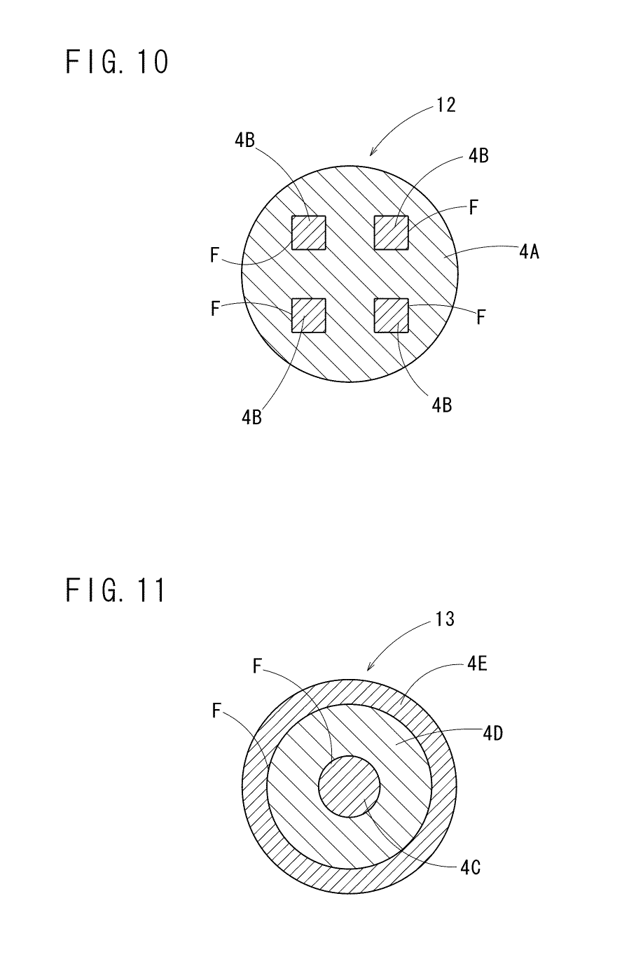

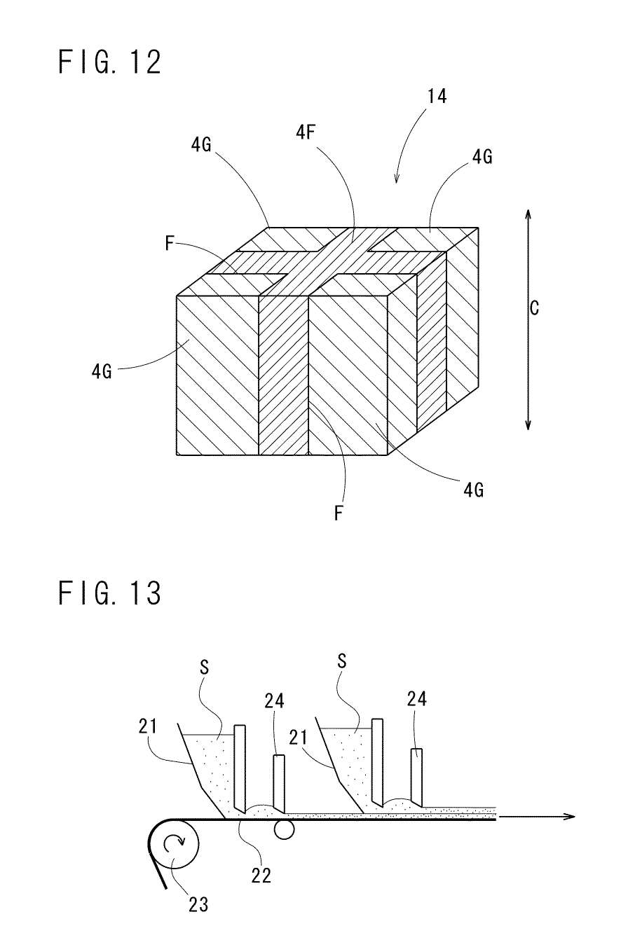

When a plurality of porous metal bodies are bonded, various configurations may be carried out as shown in FIGS. 9 to 12 besides the configurations in which the plate-shape porous metal bodies are layered as the above embodiments. For example, a porous implant material 11 shown in FIG. 9 is produced of a particular porous metal body 4A and another porous metal body 4B having a columnar-shape which is arranged in a state of being fitted in the porous metal body 4A. A porous implant material 12 shown in FIG. 10 has a plurality of columnar porous metal bodies 4B with respect to that shown in FIG. 9. In a porous implant material 13 shown in FIG. 11, a plurality of porous metal bodies 4C to 4E are multiply arranged concentrically. In a porous implant material 14 shown in FIG. 12, the porous metal body 4F is formed into a cross-shaped block, and porous metal bodies 4G which are formed into rectangular blocks are combined at four corners of the porous metal body 4F. In the producing processes, the porous implant materials can be produced by winding a plate-shape porous metal body around a particular metal body, or by rounding a plate-shape porous metal body. Prolate direction of the pores is illustrated as C-direction in FIGS. 9 and 12. The prolate directions of the pores in FIGS. 10 and 11 are orthogonal to pages.

As a bonding method, a method in which the porous metal bodies are each sintered, and then assembled and diffusion-bonded, can be accepted besides the method in which the green bodies are assembled and then sintered. When compressing, the bonded bodies having the columnar configuration shown in FIG. 9 to FIG. 11 can be compressed in the radial direction by rolling the bonded bodies of the porous metal bodies. Also, the compression process can be carried out in the state of the green sheet before sintering, or after sintering.

In each case, it is important that the bonded-boundary surfaces F are parallel to the first direction. Consequently, the compressive strength in the direction parallel to the bonded-boundary surface F can be higher than the compressive strength in the direction orthogonal to the bonded-boundary surface F. Moreover, when using as an implant, another porous metal body can be added that is bonded at a bonded-boundary surface along the other direction than the direction parallel to the prolate direction of the pores (i.e., parallel to the first direction) as appropriate if the directivity of the intended strength can be maintained.

When forming the slurry into a sheet-shape by Doctor Blade Method, the green sheets can be formed in a layered state by supplying expandable slurries each in a layered state from a plurality of hoppers as shown in FIG. 13.

Furthermore, a method of decompression-foaming can be accepted besides the method of expanding and forming by Doctor Blade Method. Specifically, pores and dissolved gas are once removed from the slurry, and then the slurry is stirred while adding gas, so that expandable slurry is made into a state in which bubble nucleus of the added gas are made and distributed therein. Subsequently, the slurry including the bubble nucleus is decompressed to a prescribed pressure and maintained at pre-cooling temperature higher than freezing point and lower than boiling point of the slurry at the prescribed pressure, so that the bubble nucleus are expanded and the slurry in which volume thereof is increased by the expansion of the bubble nucleus is vacuum-freeze dried. By sintering the green body obtained as abovementioned, porous sintered body can be produced.

### INDUSTRIAL APPLICABILITY

The implant material of the present invention can be used which is implanted into a living body as an implant such as an intervertebral spacer, a dental implant and the like.

### DESCRIPTION OF REFERENCE SYMBOLS

1 porous implant material
2 skeleton
3 pore
4 porous metal body
11 to 14 porous implant material
4A to 4G porous metal body
F bonded-boundary surface (layered-boundary surface)
C axial direction

What is claimed is:

1. Porous implant material comprising a plurality of porous metal bodies which are bonded with each other at bonded-boundary surface parallel to a first direction, wherein
each the porous metal body has a three-dimensional network structure formed from a continuous skeleton in which a plurality of pores are interconnected,
a porosity rate is 50% to 92%, and
a compressive strength compressing in a direction parallel to the bonded-boundary surface is 1.4 times to 5 times of a compressive strength compressing in a direction orthogonal to the bonded-boundary surface.

2. The porous implant material according to claim 1, wherein
the porous metal bodies are foam metal made by expanding and sintering after forming expandable slurry containing metal powder and expanding agent so as to have higher metallic density at a front surface than at a center part.

3. The porous implant material according to claim 1, wherein
the pores are formed to have flat shapes which are long along a direction parallel to the bonded-boundary surface and short along a direction orthogonal to the bonded-boundary surface, and
a length along the bonded-boundary surface is 1.2 times to 5 times of a length orthogonal to the bonded-boundary surface.

4. The porous implant material according to claim 2, wherein
the pores are formed to have flat shapes which are long along a direction parallel to the bonded-boundary surface and short along a direction orthogonal to the bonded-boundary surface, and
a length along the bonded-boundary surface is 1.2 times to 5 times of a length orthogonal to the bonded-boundary surface.

5. The porous implant material according to claim 1, wherein
part of the plurality of porous metal bodies have different porosity rate from the other porous metal bodies.

6. The porous implant material according to claim 2, wherein
part of the plurality of porous metal bodies have different porosity rate from the other porous metal bodies.

7. The porous implant material according to claim 3, wherein
part of the plurality of porous metal bodies have different porosity rate from the other porous metal bodies.

8. The porous implant material according to claim 4, wherein
part of the plurality of porous metal bodies have different porosity rate from the other porous metal bodies.
